(19) United States
(12) Patent Application Publication
(10) Pub. No.: US 2006/0140873 A1
(43) Pub. Date: Jun. 29, 2006

(54) Aerosol pharmaceutical compositions

(76) Inventor: Heng Wei Chang, Menlo Park, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(21) Appl. No.: 11/023,804

(22) Filed: Dec. 27, 2004

(51) Int. Cl. A61K 31/573; A61K 31/4745; A61L 9/04; A61M 11/00

(52) U.S. Cl. 424/45; 514/291; 514/171; 128/200.23

(57) ABSTRACT

A suspension aerosol composition suitable for use in pressurized metered dose inhalers has improved post-shaking suspension stability and comprises predetermined amounts of an active pharmaceutical ingredient (API) insoluble in the composition, a propellant comprising a hydrofluoroalkane (HFA), and a pharmaceutically acceptable non-aminated C1-6 organic acid.

### FIELD OF THE INVENTION

The field of the invention is an HFA aerosol suspension having improved post-shaking suspension stability for use in metered dose inhalers.

### BACKGROUND OF THE INVENTION

Pressurized metered dose inhalers (pMDIs) are aerosol devices that rely on propellants to deliver precisely metered doses of medication to a patient's lungs. Chlorofluorocarbons have been used as the propellants since MDIs were first introduced in the mid 1950s for treating respiratory illnesses such as asthma and chronic obstructive pulmonary disease (COPD). Due to growing awareness that CFCs contributed to the depletion of stratospheric ozone, in the mid-1980s the pharmaceutical industry began an intensive search for alternatives to the CFC-propelled MDI. Hydrofluoroalkanes (HFAs) were identified as the only suitable alternative to CFC propellants. However, the replacement of CFC propellants in pharmaceutical aerosols has proven to be a difficult challenge to the pharmaceutical industry due to the differences in the chemical and physical properties of HFAs and CFCs. In particular, many of the components that were commonly used in CFC formulations to improve various properties of the formulations, such as the surfactants used to improve suspension uniformity and stability, are difficult to dissolve in HFA. Accordingly, there is a need in the pharmaceutical industry for an HFA-based suspension aerosol composition for pMDIs having good suspension stability that provides consistent and uniform dosing.

### RELEVANT LITERATURE

U.S. Pat. No. 6,743,413 discloses pharmaceutical suspension aerosol formulations containing a therapeutically effective amount of a drug and HFC 134a, HFC 227, or a mixture thereof.

Stefely, J. S., Drug Delivery Technology (2002) Vol. 2, No. 6, discloses the use of oligolactic acids to increase the solubility of a wide variety of drugs in HFA propellants.

US Pat Publ No. 20040168950 discloses methods for packaging pMDIs containing an HFA propellant. Numerous optional components are listed that can be included in the HFA formulation, including a stabilizer (glycin, glycine, alanine, valine, leucine, isoleucine, methionine, threonine, isovaline, phenylalanine, tyrosine, serine, histidine, tryptophan, proline, hydroxyproiine, arginine, ornithine, asparagine, citrulline, aspartic acid, cysteine, glutamic acid, glutamine, lysine, hydroxylysine, N-acetyl-L-cysteine, phenylalanine, trans-4-hydroxy-L-proline, tyrosine, L-aspartyl-L-phenylalanine methylester or a mixture of any of the foregoing) and an antioxidant (tocopherol, deteroxime mesylate, methyl paraben, ethyl paraben and ascorbic acid and mixtures thereof).

U.S. Pat. No. 6,569,406 describes inhaleable spray dried 4-helix bundle protein powders having minimized aggregation with stabilizing excipients including sugars, amino acids, and oligomers comprising 2 to 5 amino acids.

### SUMMARY OF THE INVENTION

The invention provides compositions for use in an aerosol inhaler, the composition comprising predetermined amounts of an active pharmaceutical ingredient (API) insoluble in the composition, a propellant comprising a hydrofluoroalkane (HFA), and a pharmaceutically acceptable non-aminated C1-6 organic acid that increases post-shaking suspension time of the API in the composition to at least 30 seconds to provide uniform dosing of the API from the inhaler over at least 30 seconds post-shaking.

In one embodiment of the invention, the organic acid is an aliphatic carboxylic acid, preferably a saturated aliphatic carboxylic acid, such as acetic acid or lactic acid.

In one embodiment of the invention, the composition further comprises a cosolvent which increases solubility of the acid in the composition, particularly wherein the cosolvent is ethanol.

In another embodiment of the invention, the composition is essentially cosolvent-free, and/or essentially surfactant-free.

In one embodiment of the invention, the post-shaking suspension time is at least 45 seconds, preferably at least 1 minute.

In one embodiment of the invention, the API is micronized.

In one embodiment of the invention, the API is albuterol sulfate, budesonide, ipratropium bromide, or combinations thereof.

In one embodiment of the invention, the HFA is HFA134a or HFA-227.

The invention also provides methods and inhalers for delivering the subject compositions, including predetermined amounts of the compositions contained in metered dose inhalers.

### DETAILED DESCRIPTION OF EMBODIMENTS OF THE INVENTION

The invention includes compositions for use in an aerosol inhaler, particularly a pressurized metered-dose-inhaler (pMDI). In one embodiment, the compositions comprise predetermined amounts of an active pharmaceutical ingredient (API) insoluble in the composition, a propellant comprising a hydrofluoroalkane (HFA), and a pharmaceutically acceptable non-aminated C1-6 organic acid that increases post-shaking suspension time of the API in the composition to at least 30 seconds to provide uniform dosing of the API from the inhaler over at least 30 seconds post-shaking.

The organic acid is pharmaceutically acceptable, and particularly well-tolerated for repeated or chronic use in pMDIs. Preferred acids are identified by the US Food and Drug Administration (FDA) Center for Food Safety and Applied Nutrition (CFSAN) Priority-based Assessment of Food Additives (PAFA) Everything Added to Food in the United States (EAFUS) list, particularly acids subject to a Generally Recognized As Safe (GRAS) Notice. The composition may comprise a single organic acid, or a combination of two or more organic acids. Exemplary organic acids which increase post-shaking suspension time of diverse APIs to provide uniform MDI dosing are shown in Table 1.

TABLE 1
Exemplary suitable and effective organic acids

| | | | |
|---|---|---|---|
| Lactic acid | 2-methyl propionic acid | Malic acid | Maleic acid |
| Acetic acid | Butanoic acid* | Tartaric acid | Fumaric acid |
| Propionic acid | Pentanoic* | Succinic acid | Oxalic acid |
| Glycolic acid | Hexanoic acid* | Malonic aicd | Glutaric acid |
| Formic acid | Citric acid | Adipic acid | Ascorbic acid |
| Benzoic acid | Glucuronic acid | | |

*indicates inclusion of isomeric forms, particularly iso-pentanoic acid, 2-methyl-pentanoic acid, 3-methyl-pentanoic acid, 4-methyl-pentanoic acid, 2-ethyl-butanoic acid and 1-hexanoic acid.

In one embodiment, the organic acid comprises the molecular formula Cₙ₁Hₙ₂Oₙ₃, wherein n1 is an integer from 1 to 6, preferably 2 to 6, n2 is an integer from 2-12, preferably 4 to 12, and n3 is an integer from 2 to 8. In alternative embodiments, the acids include a sulfur-containing substituent, such as in benzenesulfonic acid. Preferred organic acids are saturated aliphatic carboxylic acids, particularly straight chain organic acids, such as acetic acid and lactic acid. The acids should be true acids, and not zwitterions, such amino acids. However, amino acids may be present in the composition for other purposes; for example, they may be used as excipients during milling or micronization of the API to improve particle properties. Typically, the amount of organic acid in the composition is in the range of 0.001% to 10%, preferably in the range of about 0.01% to 5%, and most preferably in the range of about 0.1% to 2%. Unless indicated otherwise, amounts disclosed herein are in parts by weight, molecular weight is average molecular weight, temperature is in degrees Centigrade, and pressure is ambient, i.e., at or near atmospheric.

The organic acids are soluble in HFA and improve the suspension stability of the API in the HFA propellant. In some embodiments, the solubility of the organic acids can be advantageously increased by the addition of a co-solvent, such as ethanol, to the composition. However, in other formulations, it may be desirable to avoid co-solvents because, for example, they may also increase drug solubility, which can negatively affect drug particle size stability via Ostwald ripening (affecting dose consistency), and reduce the respirability of the formulation (Stefely, J. S., 2002). Thus, in particular embodiments wherein a cosolvent such as ethanol is used, preferably the amount of the cosolvent in the composition is less than 5%, and more preferably, less than 1%. In some embodiments, the composition is essentially cosolvent-free.

Minute quantities of water may also be advantageously present in particular embodiments of the invention, which we have found can improve formulation dispersion and delivery. In such embodiments, the water content is typically less than 1%, more preferably less than 0.1%, preferably less than 0.01%.

The organic acid increases post-shaking suspension time of the API in the composition to at least 30 seconds to provide uniform dosing of the API from the inhaler over at least 30 seconds post-shaking. Preferred acids increase post-shaking suspension time of the API in the composition to at least 45 seconds, and preferably at least 1 minute, compared to control compositions lacking the organic acid. For example, if a composition comprising an API, lactic acid, and an HFA, has a post-shaking suspension time of 40 seconds, and a corresponding control formulation comprising identical amounts of the API and HFA (and any other component), but lacking lactic acid, has a post-shaking suspension time of 20 seconds, then the composition is considered to comprise an amount of lactic acid that increases post-shaking suspension time of the API in the composition to at least 30 seconds. In alternative embodiments, the acid increase post-shaking suspension time by at least 20 seconds, preferably by at least 30 seconds, more preferably by at lease 1 min. In alternative embodiments, the acid increases post-shaking suspension time by at least 50%, preferably by at least 100%, more preferably by at least 200%. For testing purposes, the shaking should be consistent and sufficient to suspend the API in the composition.

Post-shaking suspension time can be assessed visually using methods known in the art (e.g. Tzou, T. Z. et al., 1997). For example, the composition can be formulated or filled in glass bottles that are the same size as the inhaler container intended for use. The bottles are shaken vigorously, and suspension sedimentation is observed for one minute. From visual inspection, a milk-like suspension where the drug particles do not settle or cream for 30 seconds after shaking is considered a good suspension. A suspension is also considered desirable if it forms a flocculated 3-dimensional network where the flocculates occupy most of the volume of the suspension and separate slowly into loose sediments and supernatants (Tzou, T. Z. et al, 1997). A suspension is considered a poor formulation when particles settle (or cream) to form a supernatant and a compact sediment within 30 seconds of shaking.

Increasing post-shaking suspension time of the API in the composition provides uniform dosing of the API from the inhaler over at least 30 seconds post-shaking, preferably at least 45 seconds post-shaking, more preferably at least 1 min post-shaking. Hence, post-shaking suspension time can also be inferred from quantitative assessment of dosage uniformity, for example, by using Spray Content Uniformity (SCU) testing guidelines (FDA Center for Drug Evaluation and Research (CDER) Guidance for Industry: Nasal Spray and Inhalation Solution, Suspension, and Spray Drug Products—Chemistry, Manufacturing, and Controls Documentation, July 2002). The SCU test is demonstrates the uniformity of medication per spray, consistent with the label claim, throughout the use of the inhaler. Ten sprays are discharged from the inhaler at the beginning of use (i.e. when the inhaler container is full), and ten sprays are discharged towards the end of the intended use of the inhaler (i.e. when the inhaler container is nearly empty). The amount of active ingredient discharged from each of the sprays is determined by quantitative analysis. A composition has an acceptable SCU if (1) the amount of active ingredient per determination is not outside of 80 to 120 percent of label claim for more than 2 of 20 determinations (10 from beginning and 10 from end) from 10 containers, (2) none of the determinations is outside of 75 to 125 percent of the label claim, and (3) the mean for each of the beginning and end determinations are not outside of 85 to 115 percent of label claim. In the adapted SCU test, the sprays tested are discharged 30 seconds after vigorously shaking the inhaler. Formulations that pass SCU testing have a post-shaking suspension time of the API in the composition of at least 30 seconds.

The compositions of the invention comprise a predetermined amount of an active pharmaceutical ingredient (API) insoluble in the composition. These drugs are sufficiently insoluble such that the compositions will not deliver effective and uniform dosages without creating a suspension of the drug in the composition. Commonly, these drugs will have negligible solubility in the compositions.

Any API that has a therapeutic effect via pulmonary delivery can be used in the composition; suitable drugs include corticosteroids such as mometasone, hydrocortisone, fludrocortisone, dexamethasone, prednisone, cortisone, aldosterone, betametasone, beclomethasone, triamcinolone, budesonide, fluticasone, flunisolide; anticholinergic agents such as ipratropium bromide and tiotropium bromide; leukotriene inhibitors, including montelukast, zafirlukast and zileuton; beta2 adrenergic agonsists such as albuterol, albuterol sulfate, formoterol, salmeterol, metaproterenol, terbutaline, pirbuterol, and bitolterol (tomalate); antihistamines such as cromolyn sodium, cimetidine and clemastine; non-steroidal anti-inflammatory agents such as acetaminophen, ibuprofen, sodium nedocromil and sodium chromoglycate; anti cancer agents such as doxorubicin and fluorouracil; bronchodilators such as theophylline; antibiotics such as tobramycin and promixin; antiviral agents such as zanamivir and ribavirin; and any pharmaceutically acceptable salts, esters, hydrates, solvates, and geometric or optical isomers of the foregoing. Other APIs compatible with pulmonary delivery can be used, including as anti-tumor agents, growth hormones, heparin, insulin, interferons, interleukins, cyclosporin, leuprolide, and Dnase. The composition may contain a single API, or two or more APIs. In one embodiment, the API is albuterol sulfate, budesonide, ipratropium bromide, or combinations thereof. The compositiosn may contain a single API, or two or more APIs.

The API can be prepared as a particulate using known methods, such as by spray-drying (Steckel, H., 2004), micronization (Berry J., et al, 2004), emulsion and thermal stabilization (Tian, Y., et al, 2004), etc. The API particles are then formulated with the liquid components (i.e. HFA, C1-6 organic acid, and any other component). Alternatively, the API may be milled “in situ” with the HFA and organic acid, and any other component of the formulation (Green et al., 2004). In a preferred embodiment of the invention, the API is micronized to form drug particles of an aerodynamic size suitable for inhalation into the lungs. Preferably, at least 90% of the drug particles have a diameter of less than 6 microns, and more preferably, less than 4.7 microns. Surfactants may be used in the formulation to prevent API particles from agglomerating so that they can be dispersed from the inhaler container at an acceptable size (e.g. 1-5 microns). However, with the compositions of the invention, acceptable API particle size can be achieved without the use of surfactants. Thus, in one embodiment, the composition is essentially surfactant-free. Other components may be included in the composition such as taste masking agents, co-solvents, moisture absorbants, and anti-oxidants. Those skilled in art can substitute alternative suitable organic acids and/or include other suitable excipients, such itemized in government and industry sanctioned compendia, e.g. www.accessdata.fda.gov/scripts/cder/iig/index.cfm, and www.medicinescomplete.com/mc/excipients/current/noframes/index.htm.

In some embodiments, it is desirable to avoid direct contact between the API and the organic acid during preparing the composition. For example, the API can be placed on one side of the bottom of the inhaler container, and the organic acid on the opposite side, before capping the container with a cap with a nozzle and pressure-filling the container with a predetermined amount of HFA. Alternatively, the organic acid can be premixed with the HFA, and then pressure-filled into a container pre-filled with a predetermined amount of the API. Thus, one aspect of the invention is a composition useful as a diluent for an API in an aerosol inhaler, the composition comprising a propellant comprising a hydrofluoroalkane (HFA), and a pharmaceutically acceptable non-aminated C1-6 organic acid that increases post-shaking suspension time of the API in the composition to at least 30 seconds to provide uniform dosing of the API from the inhaler over at least 30 seconds post-shaking. In another method, all components can be combined and mixed using for example, a conventional mixer or homogenizer, by shaking, ultrasonic energy, etc. Bulk formulation can be transferred to small individual aerosol vials using for example, valve-to-valve transfer methods, pressure filling, or by using conventional cold-fill methods.

MDI canisters equipped with conventional valves, preferably metered dose valves, can be used to deliver the compositions of the invention. Routine selection of suitable MDI canister and valve assemblies (e.g. anodized or unanodized, epoxy coated or uncoated, etc.) can be made by one of ordinary skill in the art. Pressurized metered dose inhalers containing a predetermined amount of the composition of the invention are labeled according to FDA requirements to list active ingredient(s), directions regarding shaking, number of doses/container, etc. Guidance for manufacturing MDIs is well-known in the art, e.g. Guidance for Industry; Nasal Spray and Inhalation Solution, Suspension, and Spray Drug Products—Chemistry, Manufacturing, and Controls Documentation; U.S. Department of Health and Human Services, Food and Drug Administration; Center for Drug Evaluation and Research (CDER); July 2002.

### EXAMPLES

In the following examples and above, unless indicated otherwise, parts are parts by weight, molecular weight is average molecular weight, temperature is in degrees Centigrade, and pressure is ambient, i.e., at or near atmospheric.

Formulations Containing Single Active Pharmaceutical Ingredient

These formulations contain a single therapeutic component (the active pharmaceutical ingredient, or API), a C1-6 organic acid, and a propellant. Predetermined amounts of a micronized active pharmaceutical (e.g., 20 mg albuterol sulfate) and a small organic acid (e.g. 100 mg acetic acid) were carefully placed in a 25 ml glass MDI container under ambient conditions (˜1 bar, 25° C., and 50% RH). Caution was taken to ensure the drug and the acid did not come in direct contact by putting the drug powder on one side of the bottom and the acid liquid on the opposite wall. The glass container was sealed with a cap with an MDI nozzle. A predetermined amount of HFA (HFA134a, HFA227, or their mixtures) was then pressure-filled into the container using a Pamasol MDI filler. This resulted in a suspension formulation. In an alternative filling protocol, the organic acid is premixed with the desired amount of HFA propellant and then pressure-filled into an MDI container pre-filled with the API.

The suspension stability of each formulation was visually judged after shaking the bottle followed by sitting untouched for up to 1 minute. Scores from 1 to 5 were given, according to the rate of suspension sedimentation. Suspension flocculation or sedimentation occurring in less than 10 seconds had a score of 1 (poor). Suspension flocculation or sedimentation occurring between 10 to 30 seconds had a score of 2 (marginal). Suspension flocculation or sedimentation occurring between 30 to 45 seconds had score of 3 (good). Suspension flocculation or sedimentation occurring between 45 seconds to 1 minute had score of 4 (very good). If no suspension separation or flocculation occurred after 1 minute, a score of 5 (perfect) was given.

Using this protocol we demonstrate increased post-shaking suspension times for a diverse panel of APIs, HFAs and organic acids. Quantitative analyses of spray content uniformity (SCU; see, e.g. FDA Guidelines, 2002, supra) demonstrate that these formulations provide uniform dosing of the API from the inhaler over at least 30 seconds post-shaking. Table 2 summarizes exemplary suspension formulations providing suspension time increases of >1 min, and uniform dosage delivery or SCU over >1 min.

TABLE 2
Panel suspension formulations 3752-3761 provide
suspension time increases (STI) of >1 min, and
uniform dosage delivery or SCU over >1 min.

| Form. # | API | HFA | Organic Acid | STI | SCU |
|---|---|---|---|---|---|
| 3752 | albuterol | HFA134a | Acetic acid | 1 min+ | 1 min+ |
| | sulfate | | | | |
| 3753 | budesonide | HFA227 | Ascorbic acid | 1 min+ | 1 min+ |
| 3754 | ipratropium | HFA134a | Propanoic acid | 1 min+ | 1 min+ |
| | bromide | | | | |
| 3755 | hydrocortisone | HFA227 | Malic acid | 1 min+ | 1 min+ |
| 3756 | albuterol | HFA134a | Tartaric acid | 1 min+ | 1 min+ |
| | sulfate | | | | |
| 3757 | albuterol | HFA227 | Succinic acid | 1 min+ | 1 min+ |
| | sulfate | | | | |
| 3758 | budesonide | HFA134a | Malonic aicd | 1 min+ | 1 min+ |
| 3759 | ipratropium | HFA227 | Adipic acid | 1 min+ | 1 min+ |
| | bromide | | | | |
| 3760 | hydrocortisone | HFA134a | Lactic acid | 1 min+ | 1 min+ |
| 3761 | albuterol | HFA227 | Glycolic | 1 min+ | 1 min+ |
| | sulfate | | | | |

Additional detailed results for acetic and lactic acids formulations containing a single active pharmaceutical ingredient are presented in Tables 3-5, below.

TABLE 3
Suspension formulations comprising
100 mg micronized albuterol sulfate

| Example # | Stabilizer | HFA | Stability |
|---|---|---|---|
| 11 | Acetic acid 100 mg | HFA227 10 g | 5 |
| 12 | Acetic acid 100 mg | HFA134a 10 g | 5 |
| 13 | Acetic acid 10 mg | HFA227 10 g | 3 |
| 14 | Acetic acid 20 mg | HFA134a 10 g | 4 |
| 15 | Lactic acid 100 mg | HFA227 10 g | 5 |
| 16 | Lactic acid 10 mg | HFA227 10 g | 3 |
| 17 | Lactic acid 20 mg | HFA227 10 g | 4 |
| 18 | Lactic acid 20 mg | HFA134a 10 g | 4 |
| 100 | No | HFA 134a 10 g | 1 |
| 101 | No | HFA 227 10 g | 2 |

TABLE 4
Suspension formulations comprising 100 mg budesonide

| Example # | Stabilizer | HFA | Stability |
|---|---|---|---|
| 21 | Acetic acid 10 mg | HFA227 10 g | 5 |
| 22 | Acetic acid 10 mg | HFA134a 10 g | 5 |
| 23 | Lactic acid 20 mg | HFA227 10 g | 4 |
| 24 | Lactic acid 20 mg | HFA134a 10 g | 4 |
| 200 | No | HFA 134a 10 g | 2 |
| 201 | No | HFA 227 10 g | 2 to 3 |

TABLE 5
Suspension formulations comprising 20
mg micronized ipratropium bromide

| Example # | Stabilizer | HFA | Stability |
|---|---|---|---|
| 31 | Acetic acid 50 mg | HFA227 10 g | 4 |
| 32 | Lactic acid 20 mg | HFA134a 10 g | 2-3 |
| 33 | Lactic acid 50 mg | HFA134a 10 g | 3-4 |
| 34 | Lactic acid 50 mg | HFA227 10 g | 3 |
| 300 | No | HFA 134a 10 g | 1-2 |
| 301 | No | HFA 227 10 g | 2 |

Formulations Containing Multiple Active Pharmaceutical Ingredients

Formulations containing multiple APIs, an organic acid, and an HFA propellant were made and suspension stability was assessed as described above. The design and results of stabilizing a suspension of 20 mg micronized albuterol sulfate and ˜3 mg micronized ipratropium bromide in 10 gm of HFA by various amount of acetic acid, lactic acid or no stabilizer are shown in Table 6.

TABLE 6
Effect of acetic acid and lactic acid concentration on combination drug
(albuterol sulfate + ipratropium bromide) suspension stability

| Example # | Stabilizer | HFA | Stability |
|---|---|---|---|
| 31 | Acetic acid 40 mg | HFA134a 10 g | 3 |
| 32 | Acetic acid 80 mg | HFA134a 10 g | 4 |
| 33 | Acetic acid 160 mg | HFA134a 10 g | 5* |
| 34 | Acetic acid 320 mg | HFA134a 10 g | 5* |
| 35 | Lactic acid 160 mg | HFA134a 10 g | 5* |
| 36 | Lactic acid 80 mg | HFA 134a 10 g | 4 |
| 37 | Lactic acid 40 mg | HFA 134a 10 g | 3 |
| 38 | Lactic acid 20 mg | HFA227 10 g | 2 |
| 39 | Lactic acid 320 mg | HFA227 10 g | 5* |
| 40 | Lactic acid 160 mg | HFA227 10 g | 5* |
| 41 | Lactic acid 80 mg | HFA227 10 g | 4 |
| 42 | Lactic acid 40 mg | HFA227 10 g | 3 |
| 300 | None | HFA 134a 10 g | 1 |

*API partially dissolved

The design and results of stabilizing a suspension of 20 mg micronized albuterol sulfate, ˜3 mg micronized ipratropium bromide, and 40 mg budesonide in 10 ml of HFA34a by various amount of acetic acid, lactic acid or no stabilizer are shown in Table 7.

TABLE 7
Effect of acetic acid and lactic acid concentration on
combination drug (albuterol sulfate + ipratropium
bromide + budesonide) suspension stability

| Example # | Stabilizer | HFA | Stability |
|---|---|---|---|
| 1 | None | HFA134a 10 g | 1 |
| 2 | Acetic acid 40 mg | HFA134a 10 g | 2 |
| 3 | Acetic acid 80 mg | HFA134a 10 g | 3 |
| 4 | Acetic acid 160 mg | HFA134a 10 g | 4 |
| 5 | Acetic acid 320 mg | HFA134a 10 g | 4* |
| 12 | lactic acid 40 mg | HFA134a 10 g | 2 |
| 13 | lactic acid 80 mg | HFA134a 10 g | 2-3 |
| 14 | lactic acid 160 mg | HFA134a 10 g | 3 |
| 15 | lactic acid 320 mg | HFA134a 10 g | 4 |

*API partially dissolved

### CITED LITERATURE

Berry, J. et al., “Influence of the size of micronized active pharmaceutical ingredient on the aerodynamic particle size and stability of a metered dose inhaler.” Drug Dev Ind Pharm. (2004) 30:705-714.
Green, J. et al, “Pharmaceutical Aerosols—Enhancing the Metered Dose Inhaler” Drug Delivery Technology (2004) Vol. 4, No. 6.
Stefely, J. S., “Novel Excipients for Inhalation Drug Delivery: Expanding the Capability of the MDI.” Drug Delivery Technology (2002) Vol. 2, No. 6.
Steckel H, Brandes H G, “A novel spray-drying technique to produce low density particles for pulmonary delivery.” Int J. Pharm. (2004) 278:187-195
Tian Y, et al, “Evaluation of microparticles containing doxorubicin suitable for aerosol delivery to the lungs” PDA J Pharm Sci Technol. (2004) 58:266-275.
Tzou, T. Z., “Drug Form Selection in Albuterol-Containing Metered-Dose Inhalier Formulations and Its Impact on Chemical and Physical Stability, J Pharm Sci. (1997) 86:1352-1357.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition for use in a metered dose inhaler (MDI), the composition comprising predetermined amounts of an active pharmaceutical ingredient (API) insoluble in the composition, a propellant comprising a hydrofluoroalkane (HFA), and a pharmaceutically acceptable non-aminated C1-6 organic acid that increases post-shaking suspension time of the API in the composition to at least 30 seconds to provide uniform dosing of the API from the inhaler over at least 30 seconds post-shaking.

2. The composition of claim 1 wherein the organic acid is an aliphatic carboxylic acid.

3. The composition of claim 1 wherein the organic acid is a saturated aliphatic carboxylic acid.

4. The composition of claim 1, wherein the organic is selected from the group consisting of lactic acid, 2-methyl propionic acid, malic acid, maleic acid, acetic acid, butanoic acid, tartaric acid, fumaric acid, propionic acid, pentanoic acid, succinic acid, oxalic acid, glycolic acid, hexanoic acid, malonic aicd, glutaric acid, formic acid, citric acid, adipic acid, ascorbic acid, benzoic acid and glucuronic acid.

5. The composition of claim 1 wherein the organic acid is acetic acid or lactic acid.

6. The composition of claim 1 that further comprises a cosolvent which increases solubility of the acid in the composition, wherein the cosolvent is ethanol.

7. The composition of claim 1 which is essentially cosolvent-free.

8. The composition of claim 1 which is essentially surfactant-free.

9. The composition of claim 1 wherein the post-shaking suspension time is at least 1 minute.

10. The composition of claim 1 wherein the API is micronized.

11. The composition of claim 1 wherein the API is albuterol sulfate, budesonide or ipratropium bromide.

12. The composition of claim 1 wherein the HFA is HFA134a or HFA-227.

13. The composition of claim 4, wherein the API is albuterol sulfate, budesonide or ipratropium bromide, and the HFA is HFA134a or HFA-227.

14. The composition of claim 5, wherein the API is albuterol sulfate, budesonide or ipratropium bromide, and the HFA is HFA134a or HFA-227.

15. A metered dose inhaler containing a predetermined amount of the composition of claim 1.

16. A metered dose inhaler containing a predetermined amount of the composition of claim 4.

17. A metered dose inhaler containing a predetermined amount of the composition of claim 5.

18. A metered dose inhaler containing a predetermined amount of the composition of claim 13.

19. A metered dose inhaler containing a predetermined amount of the composition of claim 14.

20. A method for delivering an active pharmaceutical ingredient (API), the method comprising the steps of activating a metered dose inhaler containing a predetermined amount of the composition of claim 1 to deliver said API.
